(19) United States
(12) Patent Application Publication
(10) Pub. No.: US 2010/0047913 A1
(43) Pub. Date: Feb. 25, 2010

(54) Colloidal Gold Single Reagent Quantitative Protein Assay

(75) Inventors: Ellis Golub, Bryn Mawr, PA (US); Gerald Harrison, Upper Darby, PA (US)

Correspondence Address: RATNERPRESTIA, P.O. BOX 980, VALLEY FORGE, PA, 19482, US

(73) Assignee: The Trustees Of The University Of Pennsylvania, Philadelphia, PA (US)

(21) Appl. No.: 12/543,788

(22) Filed: Aug. 19, 2009

Related U.S. Application Data
(60) Provisional application No. 61/090,038, filed on Aug. 19, 2008.

(51) Int. Cl. G01N 33/68

(52) U.S. Cl. 436/15; 436/86

(57) ABSTRACT

The invention provides methods for determining the concentration of polypeptides. The methods generally comprise labeling a polypeptide with a metal label such as colloidal gold and quantifiably detecting the polypeptide-metal complex; the methods are improvements over other quantification assays that use colloidal gold. The invention further provides kits for practicing the methods.

### CROSS REFERENCE TO RELATED APPLICATIONS

This application claims priority to U.S. Provisional Application No. 61/090,038, filed on Aug. 19, 2008. The contents of this provisional application are incorporated by reference herein, in their entirety and for all purposes.

### STATEMENT OF GOVERNMENT SUPPORT

Research leading to the disclosed inventions was funded, in part, with funds from the National Institutes of Health, grant numbers DE013576 and DE017323. Accordingly, the United States government has certain rights in the inventions described herein.

### FIELD OF THE INVENTION

The invention relates generally to the field of protein biochemistry. More specifically, the invention relates to quantitative assays for determining the concentration of one or more proteins in a sample.

### BACKGROUND OF THE INVENTION

Analysis of protein concentration continues to be an important activity in most biomedical research laboratories. Procedures based on the ultraviolet absorbance of proteins, or their reactions with specific reagents have been published (Lowry, OH et al. (1951) J. Biol Chem. 193:265; Layne, E. et al. (1957) Methods Enzymol. p 447; Smith, P J et al. (1985) Anal. Biochem. 150:76; and, Bradford, MM et al. (1976) Anal. Biochem. 72:248). The method described by Lowry is said to be the most cited reference in the biochemical literature (Lowry et al. (1951); and, Garfield, E (1973) Curr. Contents 5:5).

In general, these procedures require relatively large amounts of sample (μg-mg), and are biased based on the particular protein property exploited in the assay. Unbiased protein assays based on amino acid analysis are considered a standard, but typically require large amounts of protein (Sittampalam, R M et al. (1988) J. Assoc. Off. Anal. Chem. 71:833). More recently, a fluorescent procedure has been developed, with somewhat higher sensitivity than earlier procedures (Hoefelshweiger, B K et al. (2005) 344:122). But this method also suffers from the problem of consuming significant quantities of valuable sample preparations.

Colloidal gold quantification of proteins has been carried out. For example, Hunter and Hunter ((1987) Anal. Biochem. 164:430) stained proteins with colloidal gold and quantified the proteins using scanning densitometry. And, Li et al. ((1989) Anal. Biochem. 182:44) also stained proteins with colloidal gold, and quantified the proteins. The Hunter method, however, suffers from several drawbacks, including the prolonged staining time, required for sufficient staining development for detection by a densitometer, which causes a higher degree of background staining and can distort the accuracy of the results, and the use of a slot blot applicator which can cause loss of sample in the apparatus, and which produces undesirable edge effects in the analyte, as well as a less uniform staining of the analyte. Application of samples via a slot blot apparatus tends to concentrate the sample at the edge of the slot, leading to non-uniform blots. The Li method also suffers from several drawbacks, including a requirement to bake the protein sample at 100 degrees C., and the immersion of the sample in immersion oil in order to clarify the paper substrate, which leaves residual oil on the sample, thereby rendering it difficult to retain the stained blot as a record.

### SUMMARY OF THE INVENTION

The invention features methods for determining the concentration of a polypeptide analyte labeled with a colloidal gold label that are improvements over other similar methods. The improvements comprise spotting the polypeptide analyte on a substrate, contacting the polypeptide analyte with a colloidal gold label, digitally acquiring an image of the labeled polypeptide analyte, and determining the density of the labeled polypeptide analyte. Preferably, at least one of the following conditions is met: The polypeptide analyte is not contacted with the colloidal gold label for more than about 3 hours; The polypeptide analyte is not baked after the spotting step; The substrate is not immersed in immersion oil.

The substrate can be a membrane such as a nitrocellulose membrane. In some aspects, the polypeptide analyte is contacted with the colloidal gold label for not more than 2 hours. The image can be digitally acquired with a scanner.

The polypeptide analyte can be dissolved in a suitable liquid, including an aqueous or oil-based liquid, and the liquid can be spotted on the substrate. Preferably, the liquid is spotted on the substrate in a volume of not more than 2 microliters.

The methods can further comprise spotting at least one polypeptide standard on the substrate, contacting the polypeptide standard with a colloidal gold label, digitally acquiring an image of the labeled polypeptide standard, determining the density of the labeled polypeptide standard, and comparing the density of the labeled polypeptide analyte with the density of the labeled polypeptide standard. The comparing can be carried out using a computer programmed to compare the density of labeled polypeptides. The density of the labeled polypeptide analyte relative to the density of the labeled polypeptide standard can indicate the concentration of the polypeptide analyte. The polypeptide standard is preferably not contacted with the colloidal gold label for more than about 3 hours, and in some aspects, the polypeptide standard is contacted with the colloidal gold label for not more than about 2 hours.

The invention also features kits for determining the concentration of a polypeptide analyte according to the methods described and exemplified herein. The kits can comprise a colloidal gold label, a substrate, and instructions for using the kit in a method for determining the concentration of a polypeptide analyte labeled with a colloidal gold label. The kit can further comprise a polypeptide standard. The substrate can be a membrane, such as a nitrocellulose membrane.

### BRIEF DESCRIPTION OF THE DRAWINGS

FIG. 1 shows an exemplary gold spot assay. Duplicate samples (2 μl) of three proteins were spotted on nitrocellulose paper, dried at room temperature, and stained with colloidal gold suspension (6 h). The nitrocellulose was then dried and digitized. Each row in the grid is a dilution series composed of 1000 ng (A), 500 ng (B), 250 ng (C) and 125 ng (D). Rows 1 & 2 are bovine serum albumin (BSA), 3 & 4 carbonic anhydrase and 5 & 6 non-fat dry milk (Blotto).

FIG. 2 shows a time course of gold spot Assay. Triplicate samples of three concentrations of BSA were spotted on 7 identical pieces of nitrocellulose paper, and incubated with colloidal gold as described in the methods section. At the indicated times, one piece of paper was removed, dried and analyzed by densitometry. The samples contained 5.35 ng (♦), 22.5 ng (▪) and 169 ng () of BSA per dot. Each point is the mean of three determinations; the error bars indicate the standard deviation.

FIG. 3 shows a dose response of the gold spot Assay. Triplicate spots of BSA (1.5-400 ng) were analyzed by the gold spot procedure. Each point is the mean of three values; the error bars are the standard deviations. Densitometry was performed using the Epson scanner.

FIG. 4 shows gold spot dose-response curves for BSA and lysozyme. Gold spot assays were carried out with serial dilutions of BSA () and lysozyme (∘) 62.5-1000 ng/spot. The amount of protein spotted was determined by dry weight. The integrated spot intensity is plotted as a function of protein amount. The points represent the mean of duplicate dots, and the error bars the standard deviation. The lines are derived from a linear regression analysis of the data.

### DETAILED DESCRIPTION OF THE INVENTION

Various terms relating to the methods and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise. Thus, for example, reference to “a polypeptide analyte” includes two or more polypeptides, including combinations or mixtures, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Polypeptide” refers to any peptide or protein comprising two or more amino acids joined to each other by peptide bonds or modified peptide bonds, i.e., peptide isosteres. Polypeptide refers to both short chains, commonly referred to as peptides, oligopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural posttranslational processes or may be made by synthetic methods.

In accordance with the present invention, the possibility that the use of colloidal gold to stain proteins on nitrocellulose Western blots could be used to quantitatively estimate protein concentration was investigated. It was observed that the concentration of a protein in a sample could be quantifiably detected with high precision and nanogram sensitivity using colloidal gold, without the need to use a slot blot apparatus to apply the sample to the nitrocellulose, without the need for extended staining time, without the need to heat the sample on the nitrocellulose, and without the need to use immersion oil to visualize the stained samples. The inventive methods are advantageous over slot blot methods because they avoid the edge effects on the sample induced by the slot blot application, including the concentration of the sample at the edge of the blot and concomitant non-uniform application of the analyte. Even if a template is used to guide sample application in the inventive methods, little or no sample solution actually contacts the edge, so little or no sample is lost on the edge (as may occur with slots) and the sample does not “bleed” under the template, which could cause further sample loss and a distorted or smudged dot.

The assay is advantageous over other procedures commonly used because it consumes very little sample, thus facilitating application of other analyses with the sample. In addition, the assay utilizes stable materials commonly available in the laboratory. Accordingly, the invention features methods for determining the concentration of a polypeptide analyte comprising labeling a polypeptide with a metal label, and quantifiably detecting the label bound to the polypeptide. The inventive methods are improvements over other quantitative protein assays that utilize metal labels such as colloidal gold.

In some aspects, methods comprise spotting a polypeptide analyte on a substrate, contacting the polypeptide analyte with a colloidal metal label for not more than about 3 hours, digitally acquiring an image of the labeled polypeptide analyte, and determining the density of the labeled polypeptide analyte, for example, determining the density of the labeled polypeptide as represented in the digital image.

The methods are suitable for analysis of any size polypeptide. The polypeptide can be monomeric or multimeric. The polypeptide can be in a native conformation, or can be fully or partially denatured. The polypeptide can be reduced.

The methods are suitable for determining the concentration of the polypeptide at the nanogram level, microgram level, or milligram level. Preferably, the methods can determine a concentration of polypeptide ranging from about 1 nanogram to about is 1 milligram, although lesser or greater concentrations can be determined. More preferably, the methods can be used to determine a concentration of polypeptide ranging from about 1 nanogram to about 10 micrograms. More preferably, the methods can be used to determine a concentration of polypeptide ranging from about 1.5 nanograms to about 1.5 micrograms. In some detailed aspects, the methods can be used to determine a concentration of a polypeptide analyte from about 1.5 nanograms to about 100 nanograms. In some detailed aspects, the methods can be used to determine a concentration of a polypeptide analyte from about 100 nanograms to about 400 nanograms.

The polypeptide to be labeled according to the inventive methods can be a polypeptide of an unknown concentration or a known concentration. The polypeptide of a known concentration can be used as a polypeptide standard. Thus, in some aspects, the methods further comprise labeling at least one polypeptide standard with the colloidal metal label, digitally acquiring an image of the labeled polypeptide standard, and determining the density of the labeled polypeptide standard, for example, the density of the labeled polypeptide as represented in the digital image. Upon determining the density of the labeled polypeptide standard, the determined density of the labeled polypeptide analyte can be compared with the density of the labeled polypeptide standard, and the density of the labeled polypeptide analyte relative to the density of the labeled polypeptide standard preferably indicates the concentration of the polypeptide analyte.

In some aspects, the methods can be carried out in solution. Thus, for example, the polypeptide (analyte and/or standard) can be mixed with a colloidal metal label to allow the metal label to attach to the polypeptide. Unbound metal label can be removed from the solution, and the labeled polypeptide remaining in solution can then be quantifiably detected. The solution can be any solution suitable for maintaining the polypeptide in a dissolved state, for example, a hydrophobic solution or an aqueous solution. The stringency of the solution can be increased, for example, by addition of detergents, polysaccharides, salts, and the like.

In some highly preferred aspects, the methods are carried out by immobilizing the polypeptide (analyte or standard) on a substrate. Thus, for example, the polypeptide can be spotted on the substrate. The polypeptide can be fixed or otherwise cross-linked to the substrate, for example, by means of chemical or ultraviolet radiation-mediated fixation, but need not be. The polypeptide can be labeled with the colloidal metal label before or after immobilization on the substrate. Preferably, the polypeptide is first immobilized on the substrate, and subsequently labeled with the metal label. Spotting can comprise, for example, dissolving the polypeptide in a suitable liquid solvent, and spotting the liquid containing the polypeptide onto the substrate. In highly preferred aspects, the liquid is spotted on the substrate and allowed to diffuse outward from the site of the spot, for example, to form a dot that is substantially circular or substantially elliptical in shape. In some aspects, the liquid is spotted without the use of a “slot blot” applicator. In some aspects, the polypeptide is not baked or otherwise heated to 100 degrees C. after spotting onto a substrate.

The liquid can be spotted onto the substrate in any suitable volume. Preferably, the volume within the range of about 1 microliter to about 100 microliters, although higher or lower volumes can be used, including volumes in the nanoliter range. The volume is more preferably not more than 10 microliters. The volume is more preferably not more than 5 microliters. In highly preferred aspects, the volume is about 2 microliters. Each spot preferably comprises not more than 150 ng of polypeptide.

For immobilizing the polypeptide, any substrate suitable in the art can be used. Preferably, the substrate is a membrane. Membranes capable of affixing polypeptides are known in the art and are commercially available. Non-limiting examples of preferred membranes include polyvinylidene difluoride (PVDF), nylon, and nitrocellulose, as well as hybrids thereof. Nitrocellulose membranes are highly preferred.

The spots can be arranged in a random or ordered orientation on the substrate. Preferably, the polypeptide is arranged in an ordered array of spots, or “dots.” The polypeptide spots can similarly be applied at any density to the substrate suitable to to the needs of the particular investigator. In some preferred aspects, the spots are applied about 1 cm apart from each other. The labeling of the polypeptide can proceed at any suitable temperature, for example, at room temperature, at body temperature, or at refrigerated temperatures. The labeling reaction can proceed for a sufficient time, which can range, for example, from a few seconds to overnight. Preferably, the labeling reaction proceeds for not more than about 3 hours. In some aspects, the labeling reaction proceeds for not more than about 2 hours. In some aspects, the labeling reaction proceeds for not more than about 1 hour. In some aspects, the labeling reaction proceeds for not more than about 12 hours, not more than about 10 hours, not more than about 8 hours, not more than about 6 hours, or not more than about 4 hours. The labeling reaction can occur with or without agitation. Following the labeling reaction, unbound metal label can be removed and the substrate washed according to any means suitable in the art.

The metal label can be comprised of any metal or metal compounds suitable in the art. In some aspects, the metal labels include metal oxides, metal hydroxides, or metal salts. In some preferred aspects, the metal label comprises a metal colloid.

The metal colloid can be in the solid or liquid phase. In more preferred aspects, the metal label comprises, for example, colloidal gold or colloidal silver. Colloidal gold is most preferred.

The polypeptide-metal label complex can be quantifiably detected using any means suitable in the art. Preferably, the complex is quantifiably detected using a densitometic analysis such as densitometry. Thus, the density of the labeled polypeptide can be determined. For example, the metal label permits visualization of the polypeptide to which it is bound. Where the polypeptide concentration is sufficiently high, the complex may be visible to the naked eye. For lesser concentrations of the polypeptide, more sensitive equipment can be used to enhance visualization. A polypeptide-gold complex produces a purple color proportional to the amount of polypeptide present, and the intensity of the color can be determined and quantified.

In some highly preferred aspects, the labeled polypeptide spots can be digitally acquired, for example, using a scanner prior to determining the density of the labeled polypeptide. For example, the substrate comprising labeled polypeptides can be scanned using a standard desktop scanner, or using a laser densitometer. Other means to scan the visualized complex include, but are not limited to photography, including digital photography, videography, especially digital videography, spectrophotometry, reflectance, or transmission. The means chosen can vary according to the needs of the particular investigator. Digitization of the labeled substrate can be carried out wet or dry. A given substrate can also be scanned by different instruments to maximize the type and range of data that can be determined. Preferably, the substrate is not immersed in or otherwise contacted with immersion oil or other image-enhancing oil prior to acquisition of the image.

Densitometric analysis of a scanned image allows for a computer-assisted quantification of the polypeptide analyte's concentration. Thus, for example, the density of the labeled polypeptide can be determined using a computer programmed to determine the density of colloidal metal-labeled polypeptides. In some highly preferred aspects, the quantification, e.g., density determination, is guided by comparison of the polypeptide analyte with a polypeptide standard, preferably placed on the same substrate and labeled in the same manner as the analyte. In some aspects, the density of the labeled polypeptide analyte can be compared with the density of the labeled polypeptide standard using a computer programmed to compare such densities. The comparison indicates the concentration of the polypeptide analyte relative to the standard, and since the standard has a known concentration, a close approximation, or a precise measure of the concentration of the analyte can be determined.

The labeled polypeptide spots can be quantified by determining the integrated density of the colored spot. In this method, the scanned image of the blot is subjected to a semi-automated analysis in which the operator identifies the location of the spot on the image, and specifies where background intensities should be obtained. After definition of the spot, the intensity of each pixel within the spot is summed, and the background intensity of the same number of pixels is subtracted, yielding the net integrated density of the spot.

Spot spectrophotmetry can also be used to quantitate the labeled polypeptide spots on the substrate. Alternatively, the labeled spots could be cut out, and the gold present in each spot determined optically or chemically.

Any polypeptide sample having a known concentration can be used as a standard in the inventive methods. Such polypeptides are commercially available, including but not limited to those that are described and exemplified herein. The inventive methods contemplate that once the concentration of a polypeptide analyte is determined or otherwise known, this information can be used to allow the polypeptide to serve as a standard against which other polypeptide analytes can be compared in order to determine their concentration.

Also featured in accordance with the present invention are kits for practicing the inventive methods. Accordingly, the invention provides kits for determining the concentration of a polypeptide analyte, comprising at least one metal label such as colloidal gold, at least one suitable substrate, and instructions for using the kit in a method for determining the concentration of a polypeptide analyte. The kits can optionally further comprise one or more polypeptide standards.

In the inventive kits, the substrate can be any membrane capable of affixing polypeptides such as PVDF, nylon, or nitrocellulose. The metal label can be any metal suitable of binding to and labeling a polypeptide, including metal colloids. Colloidal gold is highly preferred.

The following examples are provided to describe the invention in greater detail. They are intended to illustrate, not to limit, the invention.

### Example 1

### Reacients Used

Colloidal gold suspension was obtained from Bio-Rad Laboratories (Hercules, GA). The nitrocellulose paper (NCP) was Protran BA 45 (0.45 μm pore size), a product of Schleicher and Schuell (Keene, N.H.), and SDS was ultraPure 100% stock solution from Gibco (Grand Island, N.Y.). Bovine serum albumin (BSA), bovine erythrocyte carbonic anhydrase, egg white lysozyme and soybean trypsin inhibitor (SBTI) were all products of Sigma-Aldrich (St. Louis, Mo.). The Micro BCA Protein Assay kit was obtained from Pierce (Rockford, Ill.). The IntenSE BL Silver Enhancement Kit was obtained from GE healthcare.

### Example 2

### Gold Spot Assay

Protein samples (20 μl) were adjusted to 0.1% SDS, and heated at 80° C. for 10 min. Serial dilutions were made with 0.1% SDS such that 2 μl aliquots contained 1.5-1500 ng of protein. In addition to unknown samples, a standard curve was constructed using bovine serum albumin (BSA). All samples were spotted in duplicate on the same sheet of nitrocellulose.

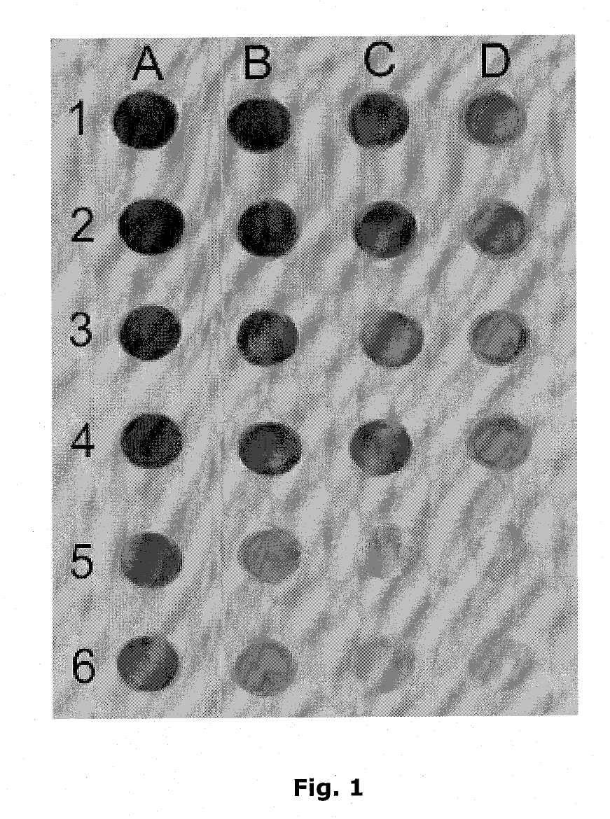

A template for guiding even rows and columns with approximately 1 cm spacing was obtained by using the flat plastic detachable 1000 μl pipette tip holder tray support from the “Space Saver” rack series (Rainin). To deposit the samples, the nitrocellulose sheet was placed on absorbent tissue paper, the template was positioned over the nitrocellulose, and was secured to the bench top with pieces of tape. Aliquots of protein sample (2 μl) were applied onto the nitrocellulose, as guided by the template, into a rectangular grid. This ordered array at 1 cm intervals provides for ample distance between dots for accurate background measurement and the use of a grid reading capability of densitometric software for rapid quantitation of the entire array for up to 96 dots per sheet (FIG. 1).

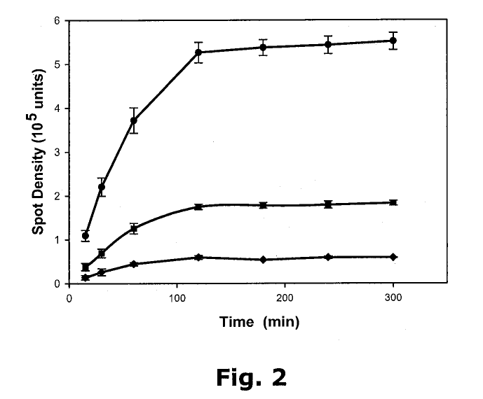

Once all standards and samples were deposited on the nitrocellulose, the array was allowed to thoroughly dry at room temperature and was then placed into the colloidal gold suspension and incubated at room temperature with gentle rocking. The staining reached an end-point after about 2 hours, although could have been left over-night, since there was no additional staining of protein dots or background after the end-point of full color development has been achieved (FIG. 2). Upon reaching the end-point, the nitrocellulose was rinsed with water and was digitized.

### Example 3

### Densitometry

Some of the data presented here was obtained using a Molecular Dynamics Personal Densitometer, and the stained spots quantified using Molecular Dynamics ImageQuant software (available from GE Healthcare). Data was also collected using an Epson Perfection 1660 PHOTO scanner, and the images were analyzed using UN-SCAN-IT-gel software (Silk Scientific, Orem Utah). While the more expensive laser densitometer was advantageous for digitizing dots at the higher end of stain density, it was observed to be too powerful for some of the lighter staining dots. The use of the Epson scanner revealed a more sensitive range for the assay, extending it down to the range of 1.5-100 ng. The measured spot intensities were transferred to Quattro Pro (Corel) or Excel (Microsoft) for statistical analysis. Graphs were prepared using SigmaPlot (Systat Software Inc.)

### Example 4

### Results

The appearance of a typical gold spot assay is shown in FIG. 1. The figure demonstrates a regular grid of gold spot 1 cm apart representing dilution series of BSA, bovine erythrocyte carbonic anhydrase and casein (Blotto) in duplicate ranging from 125-1000 ng protein, as determined by dry weight. The data in the figure show the reproducibility of the assay, and the differences in staining sensitivity amongst these proteins. To optimize the assay, the spot density for three concentrations of BSA was determined as a function of time (FIG. 2). As shown in the figure, the spot density increased with time up to about 2 hours, after which it remained stable.

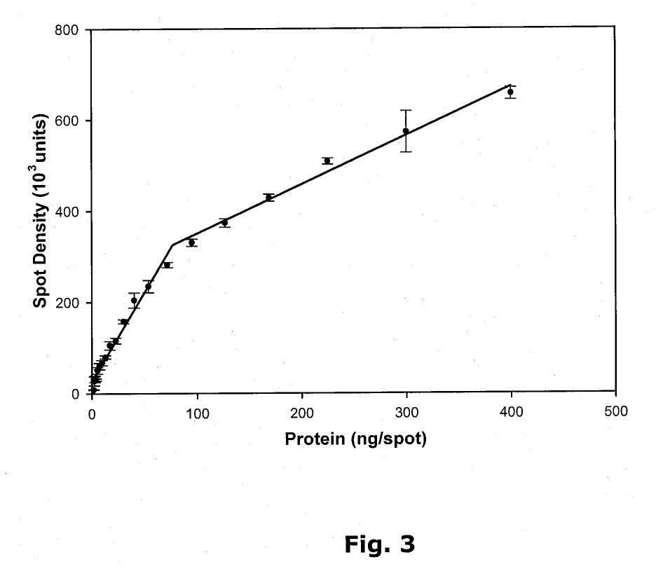

Quantitative dose-response gold spot analyses of BSA are presented in FIG. 3. Using the Epson scanner, two linear ranges of spot density were revealed: a low range (1.5-100 ng) and a higher range (100-400 ng). The upper range was further expanded through the use of the Molecular Dynamics Personal Densitometer, where spots up to 1500 ng can be measured (not shown). As is evident in the figure, the two linear ranges have different slopes. In FIG. 3, the regression lines were calculated as a 2 segment linear, piecewise fit using SigmaPlot. Alternatively, a non-linear, least squares fit to polynomial expression can be used to cover the whole range with a single expression (not shown).

The differences in densitometric measurement reflect the two different methods used. The Molecular Dynamics Densitometer measured transmitted light, while the Epson scanner measured reflected light. Thus, the lighter spots were more easily measured by the surface scanner, while the more dense, higher protein levels were better quantified by the transmitted light system. Attempts to further expand the range of the assay using the IntenSE silver enhancement kit were made. Silver enhancement had the effect of increasing the slope of the dose response curve in the lowest range, but did not extend the useful range of the assay to smaller protein samples, as the enhancement process also increased the background staining (data not shown).

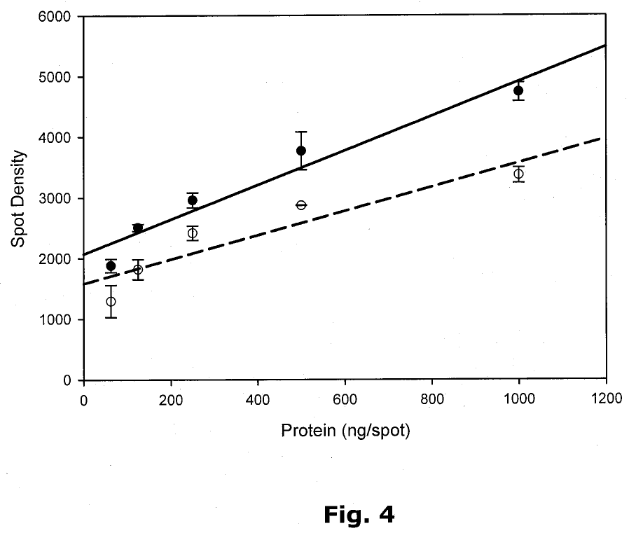

To ascertain the generality of the assay for different proteins, the staining density of BSA was compared with the staining density of lysozyme. The data in FIG. 4 clearly show that the spot density is a linear function of the amount of protein applied over the range of 100-1000 ng/spot for each protein, but the slope of the regression line is different for the two proteins. Statistical analysis of the reproducibility of the duplicate dots showed that the mean coefficient of variation (i.e., Standard Deviation/Mean) for BSA was 5%, and for lysozyme 7.6%. In the present study it was shown that the gold spot assay has a working range from 1.5-1500 ng protein/dot, with a mean coefficient of variation of 5-7%. Above 1500 ng/2 ul spot, the assay begins to saturate.

The dose response of 4 pure proteins, BSA, bovine erythrocyte carbonic anhydrase, lysozyme, and soybean trypsin inhibitor was examined, and in addition, Blotto (non-fat dry milk), which is ˜80% casein was tested. Linear relationships between gold spot intensity and the amount of protein spotted were observed for all five samples. Linear regression analysis was performed on each of the dose-response curves, and the slopes of each are shown in Table 1 as the sensitivity (Pardue, HL (1997) Clin. Chem. 43:1831).

TABLE 1
Gold spot sensitivity vs. protein molecular weight.

| | Sensitivity^{d} | Molecular | |
|---|---|---|---|
| Protein | (Spot Density/ng) | weight | pI |
| Lysozyme | 689.7 | 14,400 | 11.0 |
| SBTI^{a} | 429.9 | 21,500 | 4.5 |
| Blotto (caseins) | 620.9 | 24,000 | 4.6 |
| CA^{b} | 1096 | 29,000 | 6.0 |
| BSA^{c} | 1239 | 66,000 | 5.82 |

^{a}Soybean Trypsin Inhibitor
^{b}Carbonic Anhydrase (Bovine Erythrocyte)
^{c}Bovine Serum Albumin
^{d}Sensitivity is the slope of the dose-response regression line (see FIG. 2).

Based on the work of De Roe, et al., the slopes of the dose response curves were expected to be proportional to protein molecular weight (De Roe, C. et al. (1987) J. Histochem. Cytochem. 35:1191). But, while four of these are roughly correlated with molecular mass, the response of Lysozyme is anomalously high. While little effect of pI on the staining intensity was observed, it is conceivable that the high staining observed with lysozyme results from its high pI. Like nearly all protein assays, the instant assay was thus found to display differential sensitivity with different proteins. De Roe's previous study of protein-colloidal gold interactions in solution postulated a monomolecular shell model with the number of binding sites on a gold particle for different proteins inversely proportional to protein molecular weight. When the dose response curves for different proteins were analyzed in the gold spot assay, such a simple relationship was not observed. While the slope of the dose response curves (a measure of assay sensitivity which depends on both affinity and binding capacity) were generally proportional to protein molecular weight, two proteins, SBTI and casein, displayed an anomalously high sensitivity for their size. A better correlation was obtained between gold spot sensitivity and pI, although in this analysis, lysozyme was anomalous. These differences in assay sensitivity are similar to biases built into other protein assays. While differences in sensitivity among different proteins were observed, it is nevertheless easy to calibrate the assay for particular proteins or mixtures of proteins relative to one well behaved standard such as BSA.

Table 2 shows a comparison of the sample size requirements for the gold spot assay relative to the Pierce Micro BCA kit. While the Micro BCA assay is more rapid, it consumes considerably more sample. Recently, Noble and co-workers compared a number of dye-based spectrophotometric and fluorometric protein assays, including the BCA assay (Noble, J E et al. (2007) Mol. Biotechnol. 37:99). Based on their analysis, the gold spot assay compares favorably with the best assays in their survey in useful range, and consumes less sample. In addition, the stained nitrocellulose and digitized images provide a permanent record of the protein concentration determination.

TABLE 2
Comparison of the sample requirements and sensitivity
of the gold spot and Micro BCA protein assays

| Property | Gold Spot | Micro BCA |
|---|---|---|
| Range | 1.5-1500 ng | 0.5-25 μg |
| Sample required | 1.0 μg-10 μl | 20 μg-200 μl |
| Assay volume | 2 μl | 100 μl |

The gold spot assay was tested for interference by common reagents likely to be found in protein samples. The pH (2.8-10.3) of the sample had no effect on spot density. Glycerol (5%) and sucrose (up to 20%) did not change the spot density. At sucrose concentrations >10%, the viscosity of the sample decreased the spot diameter, but was compensated for by darker staining of the smaller dot.

Thiol reagents (dithiothreotol and mercaptoethanol) result in hyper darkening of the gold spots. While it might be possible to diminish the effects of thiol reagents on gold staining, it is believed to provide better results to assay protein content in the sample before adding the reducing agent. Because SDS was used in preparing protein samples for the analyses reported here, detergents do not appear to be a problem.

The present invention is not limited to the embodiments described and exemplified above, but is capable of variation and modification within the scope of the appended claims.

What is claimed:

1. In a method for determining the concentration of a polypeptide analyte labeled with a colloidal gold label, the improvement comprising spotting the polypeptide analyte on a substrate, contacting the polypeptide analyte with a colloidal gold label, digitally acquiring an image of the labeled polypeptide analyte, and determining the density of the labeled polypeptide analyte in the image of the labeled polypeptide analyte, wherein at least one of the following conditions is met: (a) the polypeptide analyte is contacted with the colloidal gold label for not more than about three hours, or (b) the polypeptide analyte is not baked after the spotting step.

2. The method of claim 1, wherein the substrate is a membrane.

3. The method of claim 2, wherein the membrane is a nitrocellulose membrane.

4. The method of claim 1, wherein the substrate is not immersed in immersion oil.

5. The method of claim 1, wherein the polypeptide analyte is dissolved in a liquid, and the liquid is spotted on the substrate.

6. The method of claim 5, wherein the liquid is spotted on the substrate in a volume of not more than 2 microliters.

7. The method of claim 1, wherein the polypeptide analyte is contacted with the colloidal gold label for not more than 2 hours.

8. The method of claim 1, wherein the image is digitally acquired with a scanner.

9. The method of claim 1, further comprising spotting at least one polypeptide standard on the substrate, contacting the polypeptide standard with a colloidal gold label for not more than 3 hours, digitally acquiring an image of the labeled polypeptide standard, determining the density of the labeled polypeptide standard in the image of the labeled polypeptide standard, and comparing the density of the labeled polypeptide analyte with the density of the labeled polypeptide standard; the density of the labeled polypeptide analyte relative to the density of the labeled polypeptide standard indicating the concentration of the polypeptide analyte.

10. The method of claim 9, wherein the polypeptide standard is contacted with the colloidal gold label for not more than 2 hours.

11. The method of claim 9, wherein the comparing step is carried out using a computer programmed to compare the density of a labeled polypeptide analyte with the density of a labeled polypeptide standard.

12. A kit for determining the concentration of a polypeptide analyte, comprising a colloidal gold label, a substrate, and instructions for using the kit in a method for determining the concentration of a polypeptide analyte labeled with a colloidal gold label according to claim 1.

13. The kit of claim 12, further comprising a polypeptide standard.

14. The kit of claim 12, wherein the substrate is a membrane.

15. The kit of claim 14, wherein the membrane is a nitrocellulose membrane.

16. A method for determining the concentration of a polypeptide analyte, consisting essentially of spotting the polypeptide analyte on a substrate, contacting the polypeptide analyte with a colloidal gold label for not more than about three hours, digitally acquiring an image of the labeled polypeptide analyte, and determining the density of the labeled polypeptide analyte in the image of the labeled polypeptide analyte.

17. A method for determining the concentration of a polypeptide analyte, consisting of spotting the polypeptide analyte on a substrate, contacting the polypeptide analyte with a colloidal gold label for not more than about three hours, digitally acquiring an image of the labeled polypeptide analyte, and determining the density of the labeled polypeptide analyte in the image of the labeled polypeptide analyte.
